(19) United States
(12) Patent Application Publication
(10) Pub. No.: US 2023/0288393 A1
(43) Pub. Date: Sep. 14, 2023

(54) MONITORING AND CONTROLLING BACTERIOPHAGE PRESSURE

(71) Applicant: DSM IP ASSETS B.V., Heerlen (NL)

(72) Inventors: Leonor GUEDES DA SILVA, ECHT (NL); Renger JELLEMA, ECHT (NL)

(21) Appl. No.: 18/041,362

(22) PCT Filed: Aug. 12, 2021
(86) PCT No.: PCT/EP2021/072482

§ 371 Date: Feb. 10, 2023

(30) Foreign Application Priority Data
Aug. 13, 2020 (EP) 20190823.3

(51) Int. Cl. G01N 33/04; A23C 9/12

(52) U.S. Cl. CPC G01N 33/04; A23C 9/12

(57) ABSTRACT

The present invention relates to computer implemented method for monitoring and controlling bacteriophage pressure in a milk fermentation process using a bacterial culture, said method comprises the steps of: monitoring milk fermentation process performance data; monitoring a value indicative for the number of bacteriophages; submitting the milk fermentation process performance data and the value indicative for the number of bacteriophages to a model; receiving from the model an instruction to control bacteriophage pressure; and controlling the bacteriophage pressure.

### FIELD

The present invention relates to a computer implemented method for monitoring and controlling bacteriophage pressure in a milk fermentation process using a bacterial culture. Further, the present invention relates to a data processing system for monitoring and controlling bacteriophage pressure in a milk fermentation process using a bacterial culture. According to another aspect the present invention relates to a system for monitoring and controlling bacteriophage pressure in a milk fermentation process using a bacterial culture. According to another aspect the present invention relates to a computer program product configured to be run on a machine for monitoring and controlling bacteriophage pressure in a milk fermentation process using a bacterial culture.

### BACKGROUND

Fermentation in food processing is a process wherein carbohydrates are converted into organic acids or alcohol, with the use of microorganisms under anaerobic conditions. Fermentation is often performed with yeasts or bacteria as microorganisms. Almost any food product can be fermented, such as milk, olives, beans, grains, fruit such as grapes, honey, other dairy products, fish, meat and tea.

A variety of bacterial genera are used for fermentation, for example Streptococcus, Acetobacter, Bacillus, Bifidobacterium, Lactobacillus etc. Lactobacillus for example, is able to convert sugars into lactic acid, and therefore actively lowering the pH of its environment. Some Lactobacillus species can be used as starter cultures for a high variety of fermented products, such as yogurt, cheese, sauerkraut, pickles, beer, cider, kimchi, cocoa, kefir and other fermented foods.

Bacteria that are used for fermentation can be prone to bacteriophages. A bacteriophage, or just phage, is a virus that can infect and replicate within bacteria and archaea. Phages are very diverse and common, and for virtually each bacterial strain at least one phage exists that can infect said bacterial strain. Nevertheless, phages are often only able to infect and divide within a small subset of bacterial strains. The latter can be explained by either antagonistic pleiotropy, wherein adaptation to one host is advantageous but might be deleterious in another host, or when a more general mechanism of infection is chosen, phage infection efficiency is reduced. Phages can be beneficially employed as an antibacterial agent, such as in phage therapy, and have many potential applications in human medicine as well as dentistry, veterinary science, and agriculture. Because of the specificity of phages, very specific treatments can be devised.

The thermophilic lactic acid bacterium Streptococcus thermophilus is widely used as a starter culture to improve the texture and flavor of many yoghurt and cheese products (Mora et al. (2002) Genetic diversity and technological properties of Streptococcus thermophilus strains isolated from dairy products. J Appl Microbiol, 93, 278-287). Consistent predation by (bacterio)phages, however, is still a major cause of economic losses in the dairy industry worldwide—despite a growing genetic and technological knowledge of both the hosts and phages (Goh, Y J et a/(2011) Specialized adaptation of a lactic acid bacterium to the milk environment: the comparative genomics of Streptococcus thermophilus LMD-9. Microb Cell Fact, 10 Suppl 1, S22).Bacteriophages can cause detrimental effects. This is the case in for example, the dairy industry, wherein the bacteriophages can inhibit fermentation of dairy by bacteria. Bacterial fermentation to produce other fermentation products can suffer from the same consequences. An example is the fermentation of soybean with Bacillus subtilis. Because of the specificity of bacteriophages, it is possible to change one bacterial strain for another to resolve bacteriophage infection. However, it is then necessary that the second bacterial strain is not sensitive to the same bacteriophage(s), as this would not solve the problem.

US2009/0215027 discloses a method and system for measuring the acidity and/or viscosity of milk related products, where less than desired acidification and/or viscosity can be correlated to detection of bacteriophages. The method uses a color indicator which interacts with the samples and allows to capture a digital image of the color developed on the surface of said samples. The digital image is then used to calculate a digital value representing the property of the sample. US 2009.0215027 does not relate to a (computer implemented) method to monitor or control bacteriophage pressure.

There is a need in the art for improved methods for monitoring and controlling bacteriophage pressure in milk fermentation processes.

### SUMMARY OF THE INVENTION

The present invention relates to a computer implemented method for monitoring and controlling bacteriophage pressure in a milk fermentation process using a bacterial culture, said method comprises the steps of:
(a) monitoring milk fermentation process performance data;
(b) monitoring a value indicative for the number of bacteriophages;
(c) submitting the milk fermentation process performance data and the value indicative for the number of bacteriophages to a model;
(d) receiving from the model an instruction to control bacteriophage pressure; and
(e) controlling the bacteriophage pressure.

The invention further relates to a system for monitoring and controlling bacteriophage pressure in a milk fermentation process using a bacterial culture, said system includes a controller, wherein the controller is configured to operate the system to perform the steps a) to e) of the method of the invention.

The invention further relates to a computer program product configured to be run on a machine for monitoring and controlling bacteriophage pressure in a milk fermentation process using a bacterial culture, the computer program product being configured to perform the steps a) to e) of the method of the invention.

### DETAILED DESCRIPTION

Disclosed herein is a computer implemented method for monitoring and/or controlling bacteriophage pressure in a milk fermentation process using a bacterial culture, said method comprises the steps of:
(a′) receiving milk fermentation process performance data and/or a value indicative for the number of bacteriophages;
(b′) establishing, using a model, whether and/or predicting when the milk fermentation process is outside a set of milk fermentation process operating windows; and
(c′) determining an instruction to control bacteriophage pressure.

Given the benefits of the present invention for an improved method for monitoring and/or controlling bacteriophage pressure in a milk fermentation process, for dairy manufacturers, the present invention relates also to a computer implemented method for monitoring and/or controlling bacteriophage pressure in a milk fermentation process using a bacterial culture, said method comprises the steps of:
(a) monitoring milk fermentation process performance data;
(b) optionally monitoring a value indicative for the number of bacteriophages;
(c) submitting the milk fermentation process performance data and/or the value indicative for the number of bacteriophages, preferably to a model;
(d) receiving from the model an instruction to control bacteriophage pressure; and
(e) controlling the bacteriophage pressure.

The present inventors found that by the present method, a milk fermentation company or dairy manufacturer can benefit from the expert knowledge of the provider of the bacterial culture(s) on the bacteriophage susceptibility of the bacterial culture(s).

Preferably, the present method comprising steps (a) to (e) further comprises:
(a′) receiving milk fermentation process performance data and/or a value indicative for the number of bacteriophages;
(b′) establishing, using a model, whether and/or predicting when the milk fermentation process is outside a set of milk fermentation process operating windows; and
(c′) determining an instruction to control bacteriophage pressure.

Step (a′) may be performed before step (a), before step (b), before step (c) or during or as part of step (b). Step (b′) may be performed after step (c) or during or as part of step (c). Step (c′) may be performed after step (d) or during or as part of step (d) or step (e). Preferably, the present method further comprises a step of downloading the present model and/or establishing using the model whether and/or predicting when the milk fermentation process is outside a set of milk fermentation process operating windows.

The present inventors found that by the present method the bacteriophage pressure in a milk fermentation process can be monitored and/or controlled. Further, the present invention also allows to monitor and/or control the bacteriophage pressure in real-time, and/or from a location remote from the location of the milk fermentation process. This enables an improved interaction between the dairy manufacturer or production location and the model run on a server somewhere else, or in another company than in the dairy manufacturer. This is beneficial in that the supplier of the bacterial culture, having knowledge on the bacteriophage susceptibility of the bacterial culture, can monitor and give recommendations for controlling the bacteriophage pressure in a milk fermentation process of the customer, i.e. the dairy manufacturer.

Preferably, the present step (a′) of receiving milk fermentation process performance data and/or a value indicative for the number of bacteriophages is receiving by a model milk fermentation process performance data and/or a value indicative for the number of bacteriophages. It is advantageous that only the present model receives milk fermentation process performance data and/or a value indicative for the number of bacteriophages, since this information might be confidential information from a dairy manufacturer, which might be hesitant to share the information with the supplier of the bacterial culture.

In a preferred embodiment, the present step (c′) of determining an instruction to control bacteriophage pressure comprises a step of accessing a database. The main advantage of using a database is that no further experiments are necessary to determine bacteriophage sensitivities. Such databases are often drawn up by providers of bacterial cultures and comprise information regarding bacteriophage sensitivities, and compatibility between bacterial cultures.

In such a database, if information regarding bacteriophage sensitivity is available for more than two bacterial cultures or bacterial strains, an optimal combination can be chosen wherein no common sensitivities are present. When a bacterial culture comprises more than one bacterial strain, these bacterial strains are present in a certain proportion. To minimize common bacteriophage sensitivities, and thus optimize the process, common bacteriophage sensitivities of bacterial strains in high proportion in the bacterial culture should be avoided in consecutive bacterial cultures. When it is not possible to remove all common bacteriophage sensitivities in consecutive bacterial cultures, common bacteriophage sensitivities of bacterial strains in low proportion in consecutive bacterial cultures can be chosen. A model, or the present model, can be used to solve this optimization problem.

The term “bacterial culture” (also referred to as “starter” or “starter culture”) as used herein refers to a composition comprising one or more lactic acid bacteria, which are responsible for the acidification of a milk. Starter cultures compositions may be fresh (liquid), frozen or freeze-dried. Freeze dried cultures need to be regenerated before use.

As used herein, the term “lactic acid bacteria” (LAB) or “lactic bacteria” refers to food-grade bacteria producing lactic acid as the major metabolic end-product of carbohydrate fermentation. These bacteria are related by their common metabolic and physiological characteristics and are usually Gram positive, low-GC, acid tolerant, non-sporulating, non-respiring, rod-shaped bacilli or cocci. During the fermentation stage, the consumption of lactose by these bacteria causes the formation of lactic acid, reduces the pH and leads to the formation of a protein coagulum. These bacteria are thus responsible for the acidification of milk and for the texture of the fermented milk product.

In one of its embodiments, the invention provides a method as described herein, wherein the lactic acid bacteria belong to a genus chosen from the group consisting of Streptococcus spp., Lactobacillus spp., Bifidobacterium spp., Lactococcus spp., Streptococcus salivarius thermophilus, Lactobacillus lactis, Bifidobacterium animalis, Lactococcus lactis, Lactobacillus casei, Lactobacillus plantarum, Lactobacillus helveticus, Lactobacillus acidophilus and Bifidobacterium breve.

Preferably, the used bacterial culture comprises Lactobacillus delbruekii subsp. bulgaricus and Streptococcus thermophilus.

Optionally, bacterial cultures are identified by means of a readable code provided in and/or on a packaging of said bacterial cultures.

Providers of the bacterial cultures can provide the bacterial cultures in a powdered or frozen form, being packaged for easy recognition and storage. On the packaging, a readable code such as a barcode or a quick response code (QR code) can be placed. This allows for a fast recognition of the bacterial culture and suitable compatibility with other bacterial cultures can be provided upon reading said readable code, with e.g. a computer application, such as an application for a smartphone, or even with a connection to the present model. Hence a feedback loop of scanning the readable code with the present model allows for a real-time monitoring and/or controlling of phage pressure.

In a preferred embodiment, the present model is run on a server and/or in a cloud server and/or a standalone device. For example the model can be run on a server from a company and/or location that is different from the company and/or location of the milk fermentation process. Or the present model is run in a cloud server of cloud environment, with access for more than one company.

In a preferred embodiment the time interval between receiving milk fermentation process performance data and/or a value indicative for the number of bacteriophages and the determination of an instruction to control bacteriophage pressure is as short as possible in order to facilitate process economics. Said time interval may be less than one month, less than three weeks, less than two weeks, less than one week, or from 1-10 days, or from 1 hour to 5 days or from 2 hours to 2 days.

In a preferred embodiment, the present method further comprises a step of taking one or more samples from the milk fermentation process. The sample that is isolated can originate from whey, bulk starter media, bulk starter cultures, cream, milk, acidified milk, whey powder, rinse water, a swab from dairy processes, cheese or a fermented dairy product etc. Preferably, a sample is isolated at different time points during culturing with the bacterial culture. Preferably at least two samples are taken at, at least two different time points, more preferably at least three samples are taken at, at least three different time points. More preferably, the present sample(s) is taken from whey, bulk starter media, bulk starter cultures, milk, acidified milk, whey powder, rinse water, a swab from dairy processes, cheese or a fermented dairy product.

In a preferred embodiment, milk fermentation process performance data may relate to texture, (concentration of) flavor compounds, color, time, pH, temperature, specifications of bacterial culture (e.g. colony or bacterial counts), specifications of bacterial culture used in the past, origin of the milk, type of milk, composition of the milk (e.g. fat content of the milk or protein content of the milk), amount of bacterial culture, progress in time of the fermentation process, type of fermented milk product, environmental variables (e.g. seasonality, weather, environment around process), setpoints, and/or auxiliary materials.

The type of fermented milk product, is preferably chosen from yoghurt, sour milk, quark, twarog, cream cheese, soft cheese, semi-soft cheese, semi-hard cheese, hard cheese, soured cream, cultured butter, sour cream, crème fraiche, mascarpone, mozzarella, sour milk cheese, buttermilk, schmand or smetana and blue vein cheese. More preferably the present type of fermented milk product is yoghurt or cheese.

As used herein, the term “yogurt or yoghurt” refers to a fermented milk product produced by fermentation of milk by lactic acid bacteria, also known as “yogurt cultures”. The fermentation of the (added) sugars in the milk produces lactic acid which acts on the milk protein to give the yogurt its texture. The milk is for example obtained by lactic acid fermentation by means of specific thermophilic lactic acid bacteria only (i.e. Lactobacillus delbruekii subsp. bulgaricus and Streptococcus thermophilus) which are cultured simultaneously and are found to be living in the final product in an amount of at least 10 million CFU (colony-forming unit) per gram of the yoghurt. Preferably, the yogurt is not heat-treated after fermentation. Yoghurts may optionally contain other ingredients such as sugar or sweetening agents, one or more flavoring(s), cereals or nutritional substances, especially vitamins, minerals and fibers.

Yoghurt encompasses set yoghurt, stirred yoghurt, drinking yoghurt, Petit Suisse, heat treated yoghurt and yoghurt-like products. Preferably, the yoghurt is a stirred yoghurt or a drinking yoghurt. More preferably, the yoghurt is a stirred yoghurt.

The present milk can be from an animal source, e.g. cow, goat, sheep, buffalo, etc. Additionally, milk can also have a non-dairy source, such as plant milk. Examples of plant milk include almond milk, oat milk, coconut milk, rice milk, hemp milk, soy milk, etc.

In a preferred embodiment, the present instruction to control bacteriophage pressure is chosen from the group consisting of: to rotate the bacterial culture with another bacterial culture, to increase the frequency of rotations of bacterial cultures, to change to a different rotation schedule, adjust clean in place settings, adjust clean in place frequency, clean the bioreactor of the milk fermentation process, measure value indicative for the number of bacteriophages in raw materials, improve sterilization, adjust milk fermentation process parameters and adjust amount of bacterial culture.

The term ‘clean in place’ means cleaning of the interior surfaces of pipes, vessels, process equipment, filters and/or associated fitting, without disassembly.

Basically, controlling of bacteriophage pressure is preferably carried our via cleaning to increase the hygiene in the milk fermentation process and/or via rotating the bacterial culture. The present model can correlate the milk fermentation process performance data and/or a value indicative for the number of bacteriophages to the appropriate instruction to control the bacteriophage pressure and/or to bring the milk fermentation process data back within desired operating windows.

In a preferred embodiment, the present model is a statistical model or a statistical process control model. More preferably, the present model is an artificial intelligence model and/or a machine learning model. The advantage of the present models is that the model becomes a trained model over time, being able to better correlate milk fermentation process performance data and/or a value indicative for the number of bacteriophages to phage pressure to an instruction to control bacteriophage pressure. The present model gains predictive power by adding more data to the model. For example historical data from a dairy manufacturer. Another example of adding more data is to incorporate data from more than one dairy manufacturer. The present model can better predict phage pressure for a certain dairy manufacturer if another dairy manufacturer, using for example milk from the same region, also makes use of the present methods and/or model.

In a preferred embodiment, the present value indicative for the number of bacteriophages is determined by detecting and/or identifying bacteriophages in an isolated sample or in a sample. By quantifying the bacteriophages, it would be possible to quantify the risk of infection when a bacterial culture is preceded or followed by a bacterial culture, both sensitive to said quantified bacteriophage. The term “number of bacteriophages”, as used herein, may refer to the amount of individual bacteriophage microorganisms, regardless of the strain or type of bacteriophages. It is thus a method to quantify the bacteriophages and estimate the sensitivity of the bacterial cultures and bacterial strains of the set. The value indicative for the number of bacteriophages can be a measurement value of e.g. quantitation cycle (“C_{q} value”) in a qPCR assay. In this case, a predetermined threshold value would be the value corresponding to a predetermined maximum amount of bacteriophages, up until which acidification or fermentation is still efficient.

The terms “bacteriophage”, “phage” and the like, as used herein, may refer to a virus that is only able to infect and replicate within bacteria and archaea. Bacteriophages are composed of proteins that encapsulate a DNA or RNA genome, and often have a typical outlook of an icosahedral envelop head encapsulating the nucleic acid with a tail, made up of a sheath and a baseplate with fibers attached. The tail will attach to the bacterium or the archaea, and the nucleic acid is inserted via there. The host-microorganism is then forced to translate the DNA or RNA from the bacteriophage into bacteriophage components. After assembly, the host, e.g. the bacterium, is forced to release the phages and the bacterium is often destroyed in the process.

Bacteriophages are present everywhere, including in bulk starter cultures. Generally, bacterial cultures are rotated during the process, in the hope that the strains in the cultures are different so no common bacteriophage sensitivities are present.

The terms “sensitivity”, “bacteriophage sensitivity”, “phage sensitivity” and the like, as used herein, may refer to the ability of a bacterial culture or a bacterial strain to be infected by a specific bacteriophage. When bacterial cultures or bacterial strains are said to have a common bacteriophage sensitivity, or the like, it may refer to said bacterial cultures or bacterial strains being able to be attacked or infected by at least the same bacteriophage, but not necessarily all bacteriophages present in the bacterial cultures or bacterial strains. A bacteriophage attack or bacteriophage infection comprises the insertion of bacteriophage DNA or RNA into its host, here bacteria. Additionally, the bacteriophage DNA or RNA is replicated and translated by the bacterium, resulting in a large amount of said bacteriophage. These are then released to the environment, able to infect or attack other bacteria.

Common bacteriophage sensitivities between two bacterial cultures are often the result of the cultures comprising identical or almost identical bacterial strains. This results from the high specificity of bacteriophages for their respective host.

The term “rotation”, as used herein, may refer to a cycle wherein each bacterial culture is used once in the fermentation process before starting the process anew.

In a preferred embodiment, the present value indicative for the number of bacteriophages in the sample is determined by a DNA or RNA quantification method, preferably a DNA amplification method, preferably by quantitative polymerase chain reaction (qPCR).

Quantitative polymerase chain reaction (qPCR) has as advantage that it is a very quick method, thus resulting in an even faster method for detecting bacteriophage sensitivities. qPCR is also often known as real-time polymerase chain reaction (real-time PCR). Optionally, other DNA quantification methods can be used for determining the value, preferably with a DNA amplification method. PCR is a widespread method for exponentially amplifying DNA sequences. This is performed by thermal cycling: when the temperature is high (94-98° C.), the DNA strain is split into two single-stranded DNA molecules due to the breaking of hydrogen bonds between the complementary bases. When the temperature is lowered to about 50-65° C., primers are able to bind to the single-stranded DNA, resulting of annealing of the primers to the single-stranded DNA. These primers are chosen specifically for which DNA sequence needs to be multiplied. In a third part of the thermal cycle, the complementary part of the single strand DNA is added by elongating the primer, by providing the mixture with a DNA polymerase, for example Thermus aquaticus (Taq) polymerase, and free deoxyribonucleotides (dNTPs), which are to be inserted. This last step is carried out at a temperature suitable for the DNA polymerase, e.g. 75-80° C. for Taq polymerase. The cycle is started anew after this last step. Usually, this is repeated for about 20-40 times.

In qPCR, the reaction is followed during the thermal cycling, for example via the addition of a non-specific fluorescent dye that is able to intercalate with any double-stranded DNA, or via the use of sequence-specific DNA probes consisting of oligonucleotides that have been labeled with a fluorescent reporter. The latter option is only detectable after hybridization with its complementary sequence.

Optionally, the value indicative of the number of bacteriophages is measured within 2 hours after sampling, more preferably within one hour after sampling. qPCR can be used to detect bacteriophage DNA present in the sample.

Optionally, a kit for detecting and quantifying bacteriophage DNA, such as a qPCR kit, comprises an instruction manual. In one of its aspects, the instruction manual comprises instructions to extract or purify the DNA from the dairy sample. In yet another aspect, the instruction manual comprises instructions to dilute the dairy sample, preferably to dilute the dairy sample with water and even more preferably to dilute the dairy sample with tap water. Preferably, the dairy sample is diluted at least 10 times, for example by mixing 5 ml of sample with water to a total volume of 50 ml or any other equivalent which results in a dilution of the dairy sample by a factor 10.

Preferably, the different components of the kit are in a lyophilized form allowing storing at ambient temperatures.

A method for detecting and quantifying phage DNA from a lactic acid bacteria infecting phage in a dairy sample may comprise the steps of:
(i) obtaining a sample,
(ii) optionally, diluting the obtained sample, and
(iii) testing the, optionally diluted, sample with a qPCR kit as described herein.

The dairy sample can be any of the dairy samples which are described above in the context of the kit. Typically, a dairy sample is taken at a dairy manufacturer such as a cheese or yogurt manufacturer. Preferably, a sample is taken at a cheese manufacturer. Preferably the cheese manufacturer produces cheese on large scale, i.e. a manufacturer which produces at least 3000 kg cheese per year. Or alternatively, a sample is taken from a batch or fermentation vat or fermentation vessel comprising at least 50 L of material. Yet another source of the sample is a sample from a(n) (original) pack size of at least 10 kg of powder, for example whey powder.

In a further preferred embodiment, the present value indicative for the number of bacteriophages in the sample is determined by a phage plaque assay. The phage plaque assay is an easy and straightforward method to detect virulent bacteriophages, meaning phages that damage their host cell, here a bacterium. A drawback of this method is the duration of the assay, as bacteria and bacteriophages need time to grow in a petri dish or another suitable container.

Presence of bacteriophages is detected as plaques are formed that are visible with the naked eye, plaques being spots on the surface where no bacteria are growing, as the bacteriophage has infected an initial bacterium, and has spread to bacteria surrounding said initial bacterium. By testing the sample at various dilutions of bacteriophages, the initial concentration of bacteriophages can be determined via i.e. plaque forming units (PFU) in a sample.

Optionally, the value indicative for the number of bacteriophages in the sample is determined by pH measurements during culturing of the food product with the first bacterial culture.

Preferably, the step of determining a value indicative for the number of bacteriophages, preferably in the sample and preferably quantifying phage DNA from a lactic acid bacteria infecting phage in a sample is performed at the dairy manufacturer, i.e. the sample does not have to be transported to a test lab outside of the dairy factory. In this case the dairy manufacturer submits the value indicative for the number of bacteriophages to the present model.

Alternatively, the step of determining a value indicative for the number of bacteriophages, preferably in the sample is performed by a third party. For example a research laboratory. Subsequently, the present step of submitting the value indicative for the number of bacteriophages to a model can be carried out by another entity than carrying out the steps of:
(a) monitoring milk fermentation process performance data;
(b) submitting the milk fermentation process performance data; and
(c) receiving from the model an instruction to control bacteriophage pressure; and
(d) controlling the bacteriophage pressure/(operation window).

In other words, the step of submitting the value indicative for the number of bacteriophages to the model can be carried out by a research laboratory.

Alternatively, the step of determining a value indicative for the number of bacteriophages, preferably in the sample is performed by a supplier of the bacterial culture. Hence, the dairy manufacturer can transfer the sample to the bacterial culture supplier, who determines the value indicative for the number of bacteriophages, and makes the value indicative for the number of bacteriophages available for the present model.

In a preferred embodiment, the present isolated sample is provided with a readable code. Like a QR code for example. This enables traceability of samples and data, for example when a sample transferred from a dairy manufacturer to a third party determining the value indicative for the number of bacteriophages and making the value indicative for the number of bacteriophages available in combination with the information from the readable code.

Disclose herein is a data processing system for monitoring and controlling bacteriophage pressure in a milk fermentation process using a bacterial culture, said system comprising means for carrying out any of the steps (a′) to (c′).

According to another aspect, the present invention relates to a system for monitoring and controlling bacteriophage pressure in a milk fermentation process using a bacterial culture, said system includes a controller, wherein the controller is configured to operate the system to perform the steps of:
(a) monitoring milk fermentation process performance data;
(b) monitoring a value indicative for the number of bacteriophages;
(c) submitting the milk fermentation process performance data and the value indicative for the number of bacteriophages to a model;
(d) receiving from the model an instruction to control bacteriophage pressure; and
(e) controlling the bacteriophage pressure.

Also disclosed herein is a computer program product configured to be run on a machine for monitoring and controlling bacteriophage pressure in a milk fermentation process using a bacterial culture, the computer program product being configured to:
(a′) receiving milk fermentation process performance data and/or a value indicative for the number of bacteriophages;
(b′) establishing using a model whether and/or predicting when the milk fermentation process is outside a set of milk fermentation process operating windows; and
(c′) determining an instruction to control bacteriophage pressure.
   Similarly, the present invention relates to a computer program product configured to be run on a machine for monitoring and controlling bacteriophage pressure in a milk fermentation process using a bacterial culture, the computer program product being configured to:
(a) monitoring milk fermentation process performance data;
(b) monitoring a value indicative for the number of bacteriophages;
(c) submitting the milk fermentation process performance data and the value indicative for the number of bacteriophages to a model;
(d) receiving from the model an instruction to control bacteriophage pressure; and
(e) controlling the bacteriophage pressure.

The program may be in the form of source or object code or in any other form suitable for use in the implementation of the processes according to the invention.

Some embodiments may be implemented, for example, using a machine or tangible computer-readable medium or article which may store an instruction or a set of instructions that, if executed by a machine, may cause the machine to perform a method and/or operations in accordance with the embodiments.

Various embodiments may be implemented using hardware elements, software elements, or a combination of both. Examples of hardware elements may include processors, microprocessors, circuits, application specific integrated circuits (ASIC), programmable logic devices (PLD), digital signal processors (DSP), field programmable gate array (FPGA), logic gates, registers, semiconductor device, microchips, chip sets, et cetera. Examples of software may include software components, programs, applications, computer programs, application programs, system programs, machine programs, operating system software, mobile apps, middleware, firmware, software modules, routines, subroutines, functions, computer implemented methods, procedures, software interfaces, application program interfaces (API), methods, instruction sets, computing code, computer code, et cetera.

It will be appreciated that any of the aspects, features and options described in view of the method apply equally to the system and the described computer program. It will also be clear that any one or more of the above aspects, features and options can be combined.

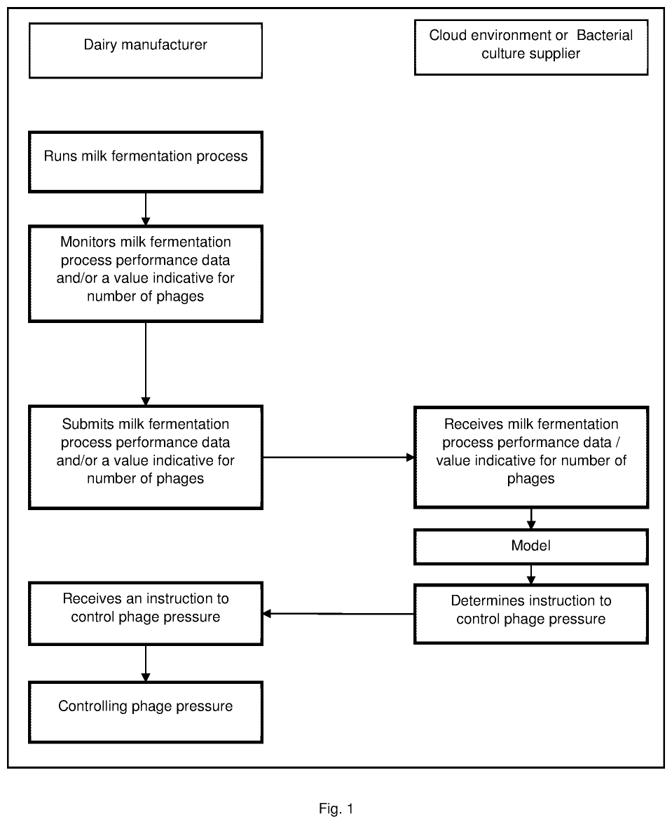

Description of exemplary embodiments of the present invention, wherein reference is made to FIGS. 1, 2 and 3.

As shown in FIG. 1, a dairy manufacturer runs a milk fermentation process using a bacterial culture. The bacterial culture can be supplied by one or more bacterial culture suppliers. The dairy manufacturer monitors milk fermentation process performance data and optionally a value indicative for the number of phages. Optionally the dairy manufacturer takes one or more samples from the milk to determine either by themselves, or by a supplier of the bacterial culture, or by another entity, a value indicative for the number of phages. The dairy manufacturer subsequently submits the milk fermentation process performance data and optionally a value indicative for the number of phages to the model. The model can be run on a server from the bacterial culture supplier, but can also be run in a cloud environment, wherein the data can be submitted without making them available for the bacterial culture supplier, but only to the present model. Subsequently the model establishes whether and/or predicting when the milk fermentation process is outside a set of milk fermentation process operating windows, for example when any delays occur in the fermentation process. Then the model determines an instruction to control the phage pressure or to bring the milk fermentation process performance data within the operating window. The dairy manufacturer receives the instruction from the model and controls the phage pressure, for example by rotating the bacterial culture and/or by increasing cleaning of the fermentation process.

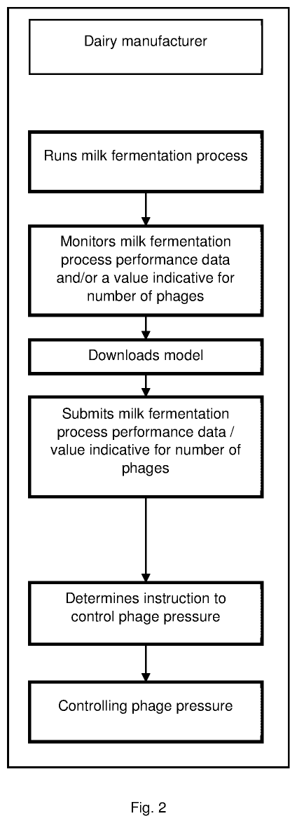

In the embodiment shown in FIG. 2, the dairy manufacturer runs through a similar process, however now the model is downloaded, for example from a supplier of the bacterial culture.

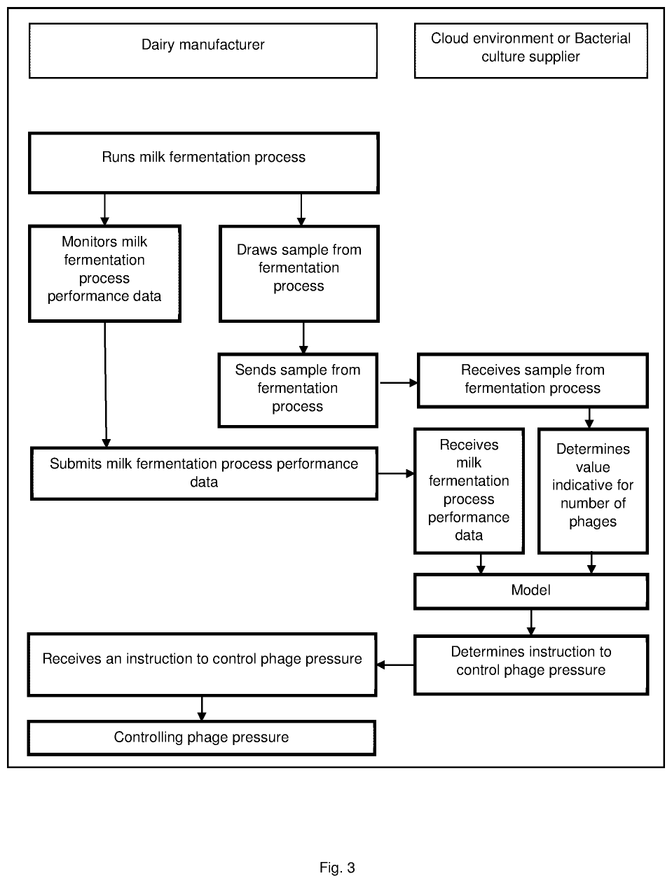

In the embodiment shown in FIG. 3, a dairy manufacturer runs through a similar process as described for FIG. 1, and now the dairy manufacturer sends a sample from the fermentation process to a supplier of bacterial cultures who determines the value indicative for the number of phages. The fermentation process performance data and the value indicative for the number of phages are submitted to the model.

1. A computer implemented method for monitoring and controlling bacteriophage pressure in a milk fermentation process using a bacterial culture, said method comprising:
(a) monitoring milk fermentation process performance data;
(b) monitoring a value indicative for a number of bacteriophages;
(c) submitting the milk fermentation process performance data and a value indicative for the number of bacteriophages to a model;
(d) receiving from the model an instruction to control bacteriophage pressure; and
(e) controlling bacteriophage pressure.

2. The method according to claim 1, wherein the model is run on a server and/or in a cloud server and/or a standalone device.

3. The method according to claim 1, further comprising taking one or more samples from the milk fermentation.

4. The method according to claim 1, wherein the milk fermentation process performance data is chosen from the group consisting of texture, flavor compounds, color, time, pH, temperature, specifications of bacterial culture, specifications of bacterial culture used in the past, origin of the milk, type of milk, composition of the milk, amount of bacterial culture, progress in time of the fermentation process, type of fermented milk product, environmental variables, setpoints, and auxiliary materials.

5. The method according to claim 1, wherein an instruction to control bacteriophage pressure is chosen from the group consisting of: to rotate the bacterial culture with another bacterial culture, to increase the frequency of rotations of bacterial cultures, to change to a different rotation schedule, adjust clean in place settings, adjust clean in place frequency, clean the bioreactor of the milk fermentation process, measure a value indicative for the number of bacteriophages in raw materials, improve sterilization, adjust milk fermentation process parameters and adjust amount of the bacterial culture.

6. The method according to claim 1, wherein the model is a statistical model or statistical process control model.

7. The method according to claim 1, wherein the model is an artificial intelligence model.

8. The method according to claim 1, wherein a value indicative for the number of bacteriophages is determined by detecting and/or identifying bacteriophages in an isolated sample.

9. The method according to claim 8, wherein the value indicative for the number of bacteriophages in the sample is determined by a DNA or RNA quantification method, optionally a DNA amplification method, optionally by quantitative polymerase chain reaction (qPCR).

10. The method according to claim 8, wherein the value indicative for the number of bacteriophages in the sample is determined by a phage plaque assay.

11. The method according to claim 8, wherein the isolated sample is provided with a readable code.

12. A system for monitoring and controlling bacteriophage pressure in a milk fermentation process using a bacterial culture, wherein said system includes a controller, wherein the controller is configured to operate the system to perform:
(a) monitoring milk fermentation process performance data;
(b) monitoring a value indicative for a number of bacteriophages;
(c) submitting the milk fermentation process performance data and a value indicative for the number of bacteriophages to a model;
(d) receiving from the model an instruction to control bacteriophage pressure; and
(e) controlling the bacteriophage pressure.

13. A computer program product configured to be run on a machine for monitoring and controlling bacteriophage pressure in a milk fermentation process using a bacterial culture, the computer program product being configured to:
(a) monitoring milk fermentation process performance data;
(b) monitoring a value indicative for a number of bacteriophages;
(c) submitting the milk fermentation process performance data and a value indicative for the number of bacteriophages to a model;
(d) receiving from the model an instruction to control bacteriophage pressure; and
(e) controlling the bacteriophage pressure.
